(19) United States
(12) Patent Application Publication
(10) Pub. No.: US 2008/0184997 A1
(43) Pub. Date: Aug. 7, 2008

(54) Anaesthetic apparatus with circulation of respiratory gases

(75) Inventor: Krzysztof Kondratowicz, WROCEAW (PL)

Correspondence Address: FURR LAW FIRM, 2622 DEBOLT ROAD, UTICA, OH, 43080, US

(73) Assignee: PPW KONTEX KRZYSZTOF KONDRATOWICZ, Tyniec Ma+hu 3+l y (PL)

(21) Appl. No.: 12/016,252

(22) Filed: Jan. 18, 2008

(30) Foreign Application Priority Data
Feb. 2, 2007 (PL) PL 384665
Oct. 25, 2007 (DE) 10 2007 054466.5.

(51) Int. Cl. A61M 16/01

(52) U.S. Cl. 128/203.12

(57) ABSTRACT

A through chamber, which functions as a circulation enforcing unit, is placed in the closed gas flow circuit. /3/. Inside the ventricle there is located a ventilator /4/. The aforementioned chamber /3/ is of cylinder shape and its axis is aligned with the axis of the ventilator's 4b rotor 4a connected with the shaft of an electric motor /4b/ with adjustable rotational speed. The through chamber /3/ has a connector /5/ joining it to the breathing bag /6/ and the anaesthetic substance evaporator /7/. The outlet of the connector /5/ is placed at the pressure side of the ventilator /4/, whereas the connecting element /8, 9/ is led out from a part of the closed circuit located between the absorber /2/ and the enforcing unit. The connecting element is an intubation tube /8/ or mask /9/ adjusted to the patient's face.

The subject of the invention is an anaesthetic apparatus with circulation of respiratory gases for inhalation anaesthesia of people and animals.

An anaesthetic apparatus with circulation of respiratory gases is known from the book by K. Ratajczak “Anastezjologia weterynaryjna”, Pańistwowe Wydawnictwo Rolnicze i Leśne, Warsaw, 1985. The apparatus was characterized by a closed circuit of gases flow, which included a carbon dioxide absorber. An evaporator containing the anaesthetic substance and breathing bag were connected to the abovementioned circuit. Between the absorber and the evaporator, an intubation tube outlet and two check valves were placed. The valves were responsible for the air flow during the patient's breath-ins and breath-outs.

### SUMMARY

The solution applied in the invention is characterized by the use of a unit enforcing gas circulation in a closed gas flow circuit. However, the unit enforcing the circulation is a through chamber with a ventilator inside. What is more, the through chamber is of cylinder shape. Its axis is aligned with the axis of the ventilator's rotor which is coupled with a shaft of an electric motor with adjustable rotational speed. Then, the through chamber is equipped with a connector for the breathing bag and anaesthetic substance evaporator; whereas, the connector's outlet is located at the pressure side of the ventilator and the connecting element is led out from a part of the closed circuit located between the absorber and the enforcing unit. Moreover, the connecting element is either an intubation tube or a mask adjusted to the patient's face.

In reference to the known state of technology, the use of an enforcing unit in a closed respiratory gas flow circuit brings a favourable effect of reducing the respiratory resistance. The advantage of the new solution applied in the invention is its easiness in adjustment to patients with various lung capacity.

### BRIEF DESCRIPTION OF THE DRAWINGS

FIG. 1 is a scheme of the anaesthetic apparatus with circulation of respiratory gases.

### DETAILED DESCRIPTION

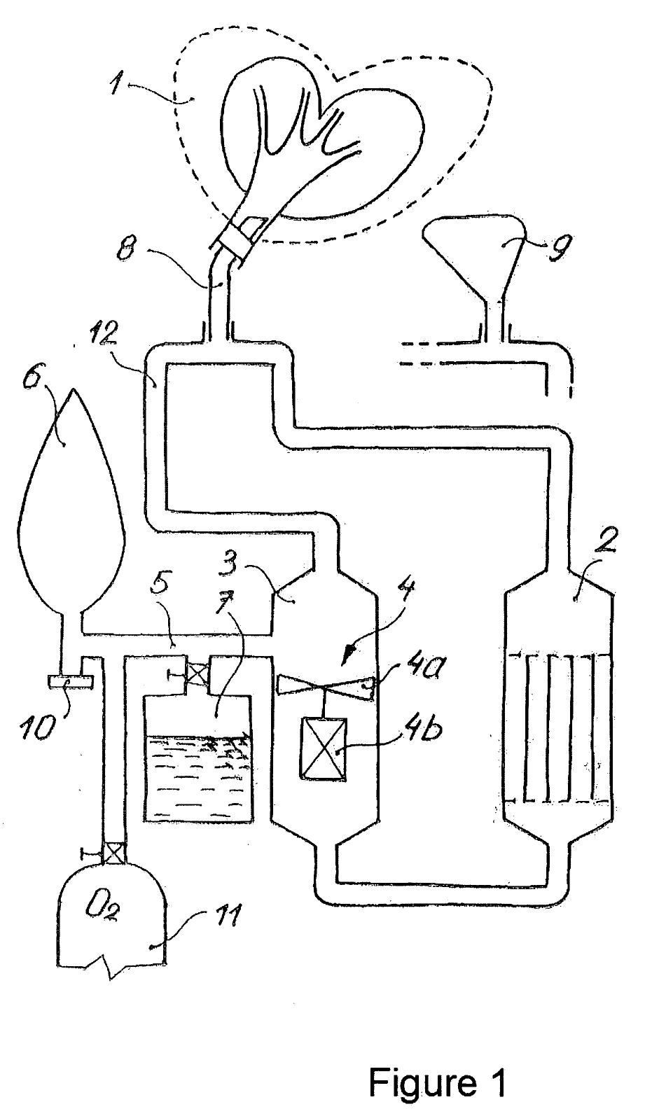

The subject of the invention was illustrated in FIG. 1, which presents the scheme of the anaesthetic apparatus with circulation of respiratory gases.

According to the invention, the anaesthetic apparatus is equipped with an element connecting to the patient's respiratory system 1, which is led out from a closed circuit, which contains a carbon dioxide absorber 2. A through chamber, which functions as a circulation enforcing unit, is placed in the closed gas flow circuit. 3. Inside the ventricle there is located a ventilator 4. The aforementioned chamber is of cylinder shape 3 and its axis is aligned with the axis of the ventilator's 4b rotor 4a, which has adjustable rotational speed. The through chamber 3 has a connector 5 joining it to the breathing bag 6 and the anaesthetic substance evaporator 7. The outlet of the connector 5 is placed at the pressure side of the ventilator 4, whereas the connecting element is led out from the closed circuit located between the absorber 2 and the enforcing unit—the through chamber 3. In the sample model of the invention the connecting element is an intubation tube 8 used interchangeably with the mask 9 adjusted to the patient's face. An overpressure valve 10, which vents the excess of respiratory gases to the atmosphere, is located at the extension of the breathing bag axis 6. The apparatus is additionally equipped with an oxygen cylinder. Its outlet is joined with the connector 5. The pipes connecting the intubation tube 8 and the mask 9 with the absorber 2 and the enforcing unit are notched pipes 12.

After connecting the apparatus to the respiratory system of the patient 1, a ventilator 4, which enforces circulation of respiratory gases, is activated. The gases have to go through the carbon dioxide absorber 2, which removes the toxic substances. Simultaneously, an anaesthetic substance necessary for correct inhalatation anaesthesia 7 of the patient is sucked in. The excess of the respiratory gases is collected in the breathing bag 6, which at the same time signals the patient's breathing process. The gases circulating in the system may be additionally enriched with oxygen from the cylinder 11.

That which is claimed is:

1. Anaesthetic apparatus with circulation of respiratory gases with a connecting element for a patient's respiratory system led out from a closed circuit, which includes a carbon dioxide absorber. The characteristic feature of the apparatus is the application of a unit which enforces gas circulation in the circuit.

2. According to claim 1, the apparatus is characteristic by the fact that the unit enforcing gas circulation is a through chamber /2/ with a ventilator /4/ placed inside.

3. According to claim 2, the apparatus is characteristic by the fact that the through chamber /2/ has a cylinder shape and its axis is in line with the axis of the ventilator's rotor /4a/. The ventilator is connected with the shaft of an electric motor /4b/ with adjustable rotational speed.

4. According to claim 3, the apparatus is characteristic by the fact that the through chamber /2/ is equipped with a connector /5/ for the through chamber /6/ and the evaporator /7/ of anaesthetic substance.

5. According to claim 3, the apparatus is characteristic by the fact that the outlet of the connector /5/ is located at the pressure side of the ventilator /4/.

6. According to claim 1, the apparatus is characteristic by the fact that the connecting element is led out from a part of the closed circuit located between the absorber /2/ and the enforcing unit.

7. According to claim 6, the apparatus is characteristic by the fact that the connecting element is an intubation tube /8/.

8. According to claim 6, the apparatus is characteristic by the fact that the connecting element is a mask /9/ adjusted to the patient's face.
